(19) United States
(12) Patent Application Publication
(10) Pub. No.: US 2025/0072820 A1
(43) Pub. Date: Mar. 6, 2025

(54) METHODS AND DEVICES FOR CONTINUOUS ORGAN AND ORGAN ALLOGRAFT MONITORING

(71) Applicant: NORTHWESTERN UNIVERSITY, Evanston, IL (US)

(72) Inventors: Lorenzo Gallon, Evanston, IL (US); John A. Rogers, Wilmette, IL (US); Surabhi Rao Madhvapathy, Evanston, IL (US); Zheng Jenny Zhang, Evanston, IL (US)

(21) Appl. No.: 18/725,885

(22) PCT Filed: Jan. 9, 2023
(86) PCT No.: PCT/US2023/010402

§ 371 Date: Jul. 1, 2024

Related U.S. Application Data
(60) Provisional application No. 63/297,331, filed on Jan. 7, 2022.

(51) Int. Cl. A61B 5/00; A61B 5/0205; A61B 5/021; A61B 5/0265; A61B 5/20

(52) U.S. Cl. CPC A61B 5/413; A61B 5/002; A61B 5/02055; A61B 5/0265; A61B 5/201; A61B 5/021

(57) ABSTRACT

The invention provides an implantable device and method of monitoring wirelessly and continuously thermal conductivity and blood flow on the surface of a target region of a subject. The implantable device comprises a probe operably attached to the target region; and an electronic module coupled with the probe for wireless, real-time, and continuous measurements of physiological information of the target region.

### CROSS-REFERENCE TO RELATED PATENT APPLICATION

This application claims priority to and the benefit of U.S. Provisional Application Ser. No. 63/297,331, filed Jan. 7, 2022, which is incorporated herein in its entirety by reference.

### FIELD OF THE INVENTION

The present invention relates generally to biosensors, and more particularly to methods and wireless implanted devices for continuous organ and organ allograft monitoring and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

End-stage renal disease (ESRD) is a major health and financial problem in the United States. Kidney transplantation is the treatment of choice for ESRD patients, however, the 5-year kidney allograft survival rate is around 72-75%. Rejection is the main cause of transplant kidney failure. Currently, there are two main diagnostic methods to evaluate kidney allograft rejection: serum creatinine and kidney biopsy. Despite the wide use of serum creatinine, it oftentimes over- or under-estimates kidney function in different conditions. Kidney biopsy is the gold standard to diagnose rejection but it carries a risk of serious complications, including bleeding, peri-renal hematoma, and arterio-venous fistula.

Therefore, a heretofore unaddressed need exists in the art to address the aforementioned deficiencies and inadequacies.

### SUMMARY OF THE INVENTION

In view of the aforementioned deficiencies and inadequacies, one of the objectives of this invention is to develop an implantable biosensor capable of monitoring wirelessly and continuously thermal conductivity and blood flow on the surface of the kidney or other organs.

In one aspect, the invention relates to a device, comprising a probe operably attached to a target region of a subject; and an electronic module coupled with the probe for wireless, real-time, and continuous measurements of physiological information of the subject.

In one embodiment, the probe is flexible.

In one embodiment, the probe comprises a temperature sensor for measuring the temperature of the target region.

In one embodiment, the probe further comprises a flow sensor for measuring the blood flow of the target region.

In one embodiment, the flow sensor comprises an optoelectronic sensor.

In one embodiment, the optoelectronic sensor comprises one or more light-emitting diodes and one or more one photodiodes.

In one embodiment, the optoelectronic sensor is a photopletismograph sensor.

In one embodiment, the probe further comprises a pressure sensor for measuring pressure.

In one embodiment, the probe further comprises a means for drug delivery.

In one embodiment, the probe has a foot print in a range of about (0.1×0.3)−(0.3×1.0) cm², a thickness in a range of about 50-500 μm, and/or Young's modulus (Y) in a range of about 30-300 kPa.

In one embodiment, the probe is constructed using thin film/wire gold encapsulated by polyimide and silicone layers.

In one embodiment, the electronic module is a flexible, miniaturized electronic module adapted for rechargeable powering, circuit control, signal processing, and wireless data communication.

In one embodiment, the electronic module comprises a flexible printed circuit board (fPCB), electronic components mounted onto the fPCB, and a power module coupled with the electronic components.

In one embodiment, the fPCB comprises a flexible substrate and conductive traces, pads and outline defined on the flexible substrate.

In one embodiment, the flexible substrate is formed of a flexible material.

In one embodiment, the power module comprises at least one battery.

In one embodiment, the at least one battery is rechargeable.

In one embodiment, the at least one battery is rechargeable via wireless power transfer.

In one embodiment, the electronic components comprise a data processing module coupled to the probe for receiving data from the probe and processing the received data, and a radio module coupled to the data processing module for wireless data transmission to an external device.

In one embodiment, the data processing module comprises a controller and an analog to digital front end.

In one embodiment, the radio module is configured to perform wireless communications using at least one communication protocol of near field communication (NFC), Wi-Fi/Internet, Bluetooth, Bluetooth low energy (BLE), and Cellular communication protocols.

In one embodiment, the radio module comprises at least one of an NFC interface and a Bluetooth interface.

In one embodiment, the external device is a mobile device, a computer, or an ICU monitoring display.

In one embodiment, the device further comprise a customized app with a graphical user interface deployed on the external device that enables real-time visualization, storage, and analysis of measured data. The graphical user interface provides a control interface to the device.

In one embodiment, the device further comprises an elastomeric encapsulation layer at least partially surrounding the probe and the electronic module.

In one embodiment, the elastomeric encapsulation layer is formed of a medical-grade, biocompatible silicone.

In one embodiment, the target region is an organ or transplanted organ.

In one embodiment, the organ or transplanted organ is a kidney, a liver, a lung, a heart or other organ.

In one embodiment, the physiological information comprises tissue temperature, thermal conductivity, and/or blood flow.

In one embodiment, the device is used for continuous, real-time monitoring of organ temperature and perfusion for detecting graft-rejection associated inflammatory processes in organ transplant intra-operatively and/or post-operatively.

In one embodiment, the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

In one embodiment, the thermal power is chosen such that ΔT<2° C.

In one embodiment, the device is mechanically compliant and water resistant.

In another aspect, the invention relates to a method for continuously monitoring a target region of a subject in real time. The method comprises attaching a device on the target region, wherein the device comprises a probe and an electronic module coupled with the probe for wireless, real-time, and continuous measurements of physiological information of the target region; measuring temperature and perfusion of the target region; and processing the measured temperature and perfusion by the electronic module to identifying a surrogate marker for detecting graft-rejection associated inflammatory processes in organ transplant intra-operatively and/or post-operatively.

In one embodiment, the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

In one embodiment, the thermal power is chosen such that ΔT<2° C.

In one embodiment, said processing the measured temperature and perfusion comprises identifying unique temperature signatures for different rejection-related biological processes/mechanisms.

In one embodiment, the temperature not only provides early warning of rejection episodes but also helps personalized dosing strategies including correct dosing, dosing regimens, and efficacy of different drugs and therapies through monitoring of the magnitude of and time between different features in the temperature including the inflection point, temperature peak, and half-day frequency.

In one embodiment, the surrogate marker is temperature variations on the surface of the target region.

In one embodiment, the method further comprises inferring a degree of damage that occurs during ischemia-reperfusion injury (IRI) and the possible recovery based on the perfusion.

In one embodiment, the method further comprises wirelessly transmitting the processed temperature and perfusion to an external device by the electronic module.

In one embodiment, the method further comprises alerting the subject and/or a physician of possible injury to the graft, based on the surrogate marker.

In one embodiment, the target region is an organ or transplanted organ.

In one embodiment, the organ or transplanted organ is a kidney, a liver, a lung a heart, or other organ.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1A shows schematically a block diagram of a device according to embodiments of the invention.

FIG. 1B shows schematically of a probe and the probe fabrication procedure of a device according to embodiments of the invention. Panel a: an exploded view of the probe. Panel b: an enlarged view of the probe. Panel c: a detailed fabrication procedure of the probe.

FIG. 1C shows schematically details of electronics of a device according to embodiments of the invention. Panels a-b: photographs of the device including the probe and electronics, Panels c-d: photographs of the electronics. Panel e: implanted sensor beacon block diagram. Panel f: data logger. Panel g: a data processing flowchart.

FIG. 2 shows schematically a soft, stretchable thermal sensor probe implanted under the kidney capsule according to embodiments of the invention.

FIG. 3 shows the surface temperatures of the kidney cortex measured by the probe in rat models according to embodiments of the invention.

FIG. 4 shows the surface temperatures of the kidney cortex measured by the probe in rat models and probe implanted under the kidney capsule, according to embodiments of the invention.

FIG. 5 shows schematically a miniaturized version of an implantable thermal sensor according to embodiments of the invention.

FIG. 6 shows proposal for immunosuppressant experiments according to embodiments of the invention.

FIG. 7 shows monitoring kidney transplant rejection using fully implantable thermal sensors according to embodiments of the invention. Panel a: Illustration of the rat kidney transplant and device implantation. The removal of both native kidneys is represented by the dashed white lines. Panel b: The soft probe directly interfaces with the cortex and is sutured to the overlying renal capsule via two suture holes. Panel c: Photograph of the device next to a US Quarter for relative size (scale bar=5 mm). Photographs of (panel d) the probe implanted on the dorsal side of the kidney and (panel e) the abdominal cavity once the kidney is returned to its final position. Panel f: False-colored 3D reconstruction of a Computerized Tomography (CT) image collected with soft tissue contrast in a representative isogeneic transplant ˜2 months after surgery. The electronics and probe remain in their original position. Panel g: Finite Element Analysis (FEA) illustrating heat distribution from the probe into the kidney during thermal anemometry-based perfusion measurements. Panel h: Continuous organ temperature (T_{kidney}) and perfusion (ω) data collected for a control Lewis rat with single native kidney (second kidney nephrectomized) for t=0-27 days.

FIG. 8 shows characterization of acute rejection according to embodiments of the invention. Illustrations of a rat kidney transplantation model using inbred rat strains for (panel a) isogeneic transplant, where both the donor and recipient strains are Lewis rats, leading to graft acceptance and (panel b) allogeneic transplant, where the donor strain is the August Copenhagen Irish (ACI) rat and the recipient is a Lewis rat, resulting in graft rejection. T_{kidney} measured for 7 days for (panel c) n=5 isografts and (panel d) n=5 allografts. In this and subsequent figures, each individual dataset is labeled with a unique animal identifier (e.g., A1-5, I1-5). The gray points represent the raw data. The red line is a smoothing spline fit (λ=0.01). The shaded region (t=0-2 days) indicates the post-surgery recovery period where a heating pad was applied underneath half of the animal cage. The black arrows in panel d correspond to a feature in T_{kidney} (bump and inflection point) unique to the allografts at t˜3 days. The red arrows in panel d correspond to a sharp decrease in T_{kidney} at t˜5 days. Representative Periodic acid-Schiff (PAS) stained histological section of an (panel e) isograft kidney harvested at t=23 days, displaying no signs of rejection and (panel f) allograft kidney harvested at t=6 days, with severe rejection characterized by Thrombotic Microangiopathy (TMA) and diffuse cortical necrosis.

FIG. 9 shows kidney temperature for advanced prediction of acute rejection according to embodiments of the invention. Raw T_{kidney} data (gray points) and smoothing spline fit (red curve, λ=0.01) for a representative ˜4 day long (panel a) isogeneic and (panel b) allogeneic transplant. The shaded region (t=0-2 days) indicates the post-surgery recovery period where a heating pad was applied underneath half of the animal cage. The dotted lines indicate the time of kidney harvest. PAS-stained histological sections show (panel c) the isograft kidney (I6) is normal at t˜4 days (corresponding data in panel a), while (panel d) the allograft kidney shows signs of Type I acute rejection at t˜4 days (corresponding data in panel b). Data in panels e-g and j are collected from the same animals with sensor data in panel c-d of FIG. 7. Panel e: Blood Urea Nitrogen (BUN) (panel f) and serum creatinine levels at t˜6 days are elevated for allografts relative to isografts. T̅ₙ represents kidney temperature averaged over t=n−1 to t=n days. Panel g: (T̅₆−T̅₅) is lower in allografts compared to isografts, denoting the steep fall in T_{kidney} marked by the red arrows in panel d of FIG. 8. (Panel h) BUN, and (panel i) serum creatinine at t˜4 days are not significantly different between allografts and isografts (panel j) (T̅₄−T̅₃) is a statistically significant metric for the feature (bump and inflection point) marked by the black arrows in panel d of FIG. 8. The green shaded region in panels e-f and h-i represent normal levels for Lewis rats without transplant.

FIG. 10 shows delaying graft rejection with immunosuppressants according to embodiments of the invention. Panel a: Illustration of the subcutaneous implantation of an osmotic pump in an allograft for delivery of FK506 (Tacrolimus) at a continuous dose of 1 mg/kg/day for t=0-7 days. Panel b: Serum creatinine and BUN collected at discrete time points (t˜4, 7, 10, 14, 20, and 27 days) for animals treated with FK506. Panel c: T_{kidney} vs. t for a representative isograft for 28 days. Panels d-h: T_{kidney} vs. t for n=5 allografts treated with 1 mg/kg/day FK506 for t=0-7 days (treatment period denoted by blue shaded region). The grey shaded region (t=0-2 days) indicates the post-surgery recovery period where a heating pad was applied underneath half of the animal cage. The gray points represent the raw data. The red curve is a smoothing spline fit (λ=0.01). The black arrows correspond to the onset of a muted temperature ‘bump.’ Panel i: Representative histological sections stained with PAS for a harvested kidney at t=10 days shows no signs of rejection, while those at t=14, 20, 27 days all show progressively increasing signs of Type I acute rejection (Type 1B rejection for the image at t=27 days).

FIG. 11 shows temperature as a reliable advanced indicator of rejection relative to blood markers according to embodiments of the invention. Panel a: T_{kidney} for a representative isograft and allograft for t=9-21 days. Gray points represent the raw data. The red curve is a smoothing spline fit (λ=30). Kidney temperature averaged over t=n−1 to t=n days is represented by T̅ₙ. T̅₁₀, T̅₁₄, and T̅₂₀ denote the rise, peak, and decline of T_{kidney} respectively. Panel b: The ‘bump’ height (T̅₁₄−T̅₁₀) and subsequent decline (T̅₂₀−T̅₁₄) are significant in medicated allografts compared to isografts (n=5). Panel c: Differences in BUN and serum creatinine between medicated allografts and isografts are not significant for t=10, 14, and 20 days. Panel d: T_{kidney} for a representative isograft and allograft for t=7-21 days. Points represent the raw data. The red curve is a smoothing spline fit (λ=0.01). Panel e: Amplitude spectra for the T_{kidney} data from panel d (for t=7-21 days) in the frequency-domain show the presence of an additional ƒ=0.5 day⁻¹ feature in medicated allografts. Panel f: The magnitude of the ƒ=0.5 day⁻¹ peak (|X₂|) normalized by that of ƒ=1 day⁻¹ (|X₁|) for t=7-14 days (top) and t=14-21 days (bottom) is greater in medicated allografts relative to isografts (n=5). Confusion matrices comparing the measured values of (panel g) BUN and (panel h) creatinine in isografts and medicated allografts to blinded histological diagnosis show 50% and 54% predictive accuracy, respectively. Confusion matrix comparing (panel i) (T̅₁₄−T̅₁₀) and (panel j) |X₂|/|X₁| to blinded histological diagnosis show predictive accuracies of 75% and 100%, respectively. The cutoff values are (panel g) the upper limits of normal BUN and Creatine for Lewis rats and (panel h) the corresponding grand means of the datasets. The red shaded regions correspond to false positive and negative evaluations, and the green shaded regions are true negative and positive outcomes.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “bidirectional wireless communication system” refers to onboard components of sensors, wireless controller and other electronic components that provides capability of receiving and sending signals using at least one communication protocol of near field communication (NFC), Wi-Fi/Internet, Bluetooth, Bluetooth low energy (BLE), and Cellular communication protocols for wireless communication. In this manner, an output may be provided to an external device, including a cloud-based device, personal portable device, or a caregiver's computer system. Similarly, a command may be sent to the sensor, such as by an external controller, which may or may not correspond to the external device. Machine learning algorithms may be employed to improve signal analysis and, in turn, command signals sent to the medical sensor, including a stimulator of the medical sensor for providing haptic signal to a user of the medical device useful in a therapy. More generally, these systems may be incorporated into a processor, such as a microprocessor located on-board or physically remote from the electronic device of the medical sensor. An example of the wireless controller is a near field communication (NFC) chip, including NFC chips. NFC is a radio technology enabling bi-directional short range wireless communication between devices. Another example of a wireless controller is a Bluetooth® chip, or a BLE system-on-chip (SoC), which enables devices to communicate via a standard radio frequency instead of through cables, wires or direct user action.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “bending stiffness” refers to a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

The terms “Young's modulus” and “modulus” are used interchangeably and refer to a mechanical property of a material, device or layer which refers to the ratio of stress to strain for a given substance. Young's modulus may be provided by the expression;

$$E = \frac{( stress )}{( strain )} = ( \frac{{L}_{0}}{Δ L} ) ( \frac{F}{A} ) ,$$

where E is Young's modulus, L₀ is the equilibrium length, ΔL is the length change under the applied stress, F is the force applied and A is the area over which the force is applied. Young's modulus may also be expressed in terms of Lame constants via the equation:

$$E = \frac{μ ( 3 λ + 2 μ )}{λ + μ}$$

where λ and μ are Lame constants. High Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably 10 times larger for some applications, more preferably 100 times larger for other applications and even more preferably 1000 times larger for yet other applications. “Inhomogeneous Young's modulus” refers to a material having a Young's modulus that spatially varies (e.g., changes with surface location). A material having an inhomogeneous Young's modulus may optionally be described in terms of a “bulk” or “average” Young's modulus for the entire layer of material.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers useful include, but are not limited to, thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to, silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. In one embodiment, a flexible polymer is a flexible elastomer.

The term “encapsulate” or “encapsulation”, as used in the disclosure, refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes devices having partially or completely encapsulated electronic devices, device components and/or inorganic semiconductor components.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Rejection is the result of complex cascades of immune system response with chemokines, cytokines, and multiple immune cells. This immune system activation leads to tissue damage, inflammation, and microvascular injury. The immune system activation can affect thermal conductivity and blood flow changes in the setting of kidney allograft rejection. Furthermore, all transplanted organs experience ischemia-reperfusion injury (IRI), and also native kidneys are subjected to IRI. IRI evokes major intra-graft inflammatory immune responses that augment graft immunogenicity, increase the rate of allograft rejection, and negatively impact solid organ transplantation outcomes. Recovery from IRI is unpredictable.

Rejection is the main cause of transplant kidney failure. Currently, there are two main diagnostic methods to evaluate kidney allograft rejection: serum creatinine and kidney biopsy. Creatinine has been used as a kidney function marker for a long time due to wide availability and low cost but has significant weaknesses including lack of accuracy, delayed increase after acute kidney injury insult, and erroneous readings from interaction with drugs and muscle mass. Also, serum creatinine requires blood from the patient for analysis.

Kidney biopsy is the gold standard to diagnose rejection but it carries a risk of serious complications, including bleeding, peri-renal hematoma, and arterio-venous fistula. A kidney biopsy cannot be used as a continuous monitoring tool but only to confirm rejection and document the severity of the rejection.

New blood biomarkers of kidney allograft rejection to replace or add to creatinine or kidney biopsy were recently discovered. Unfortunately, these biomarkers have very poor positive predictive value, are extremely costly, and cannot be used for continuous monitoring since they required blood from patients for analysis.

Therefore, developing a continuous monitoring system has great potential to improve kidney allograft survival by detecting early rejection, avoiding unnecessary kidney biopsies, and by providing real-time information, to the patient and to the physician, of the health status of the kidney transplant, continuous monitoring will allow rapid medical interventions.

The objectives of this invention is to develop an implantable biosensor/device capable of monitoring wirelessly and continuously thermal conductivity and blood flow on the surface of the kidney or other organs, so as to detect and evaluate the rejection in real time.

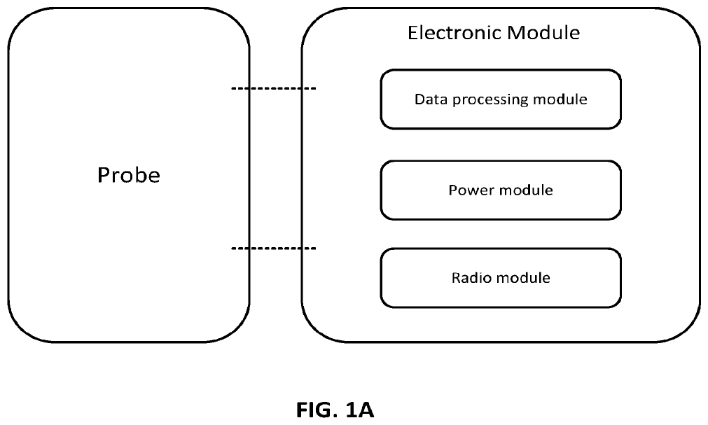

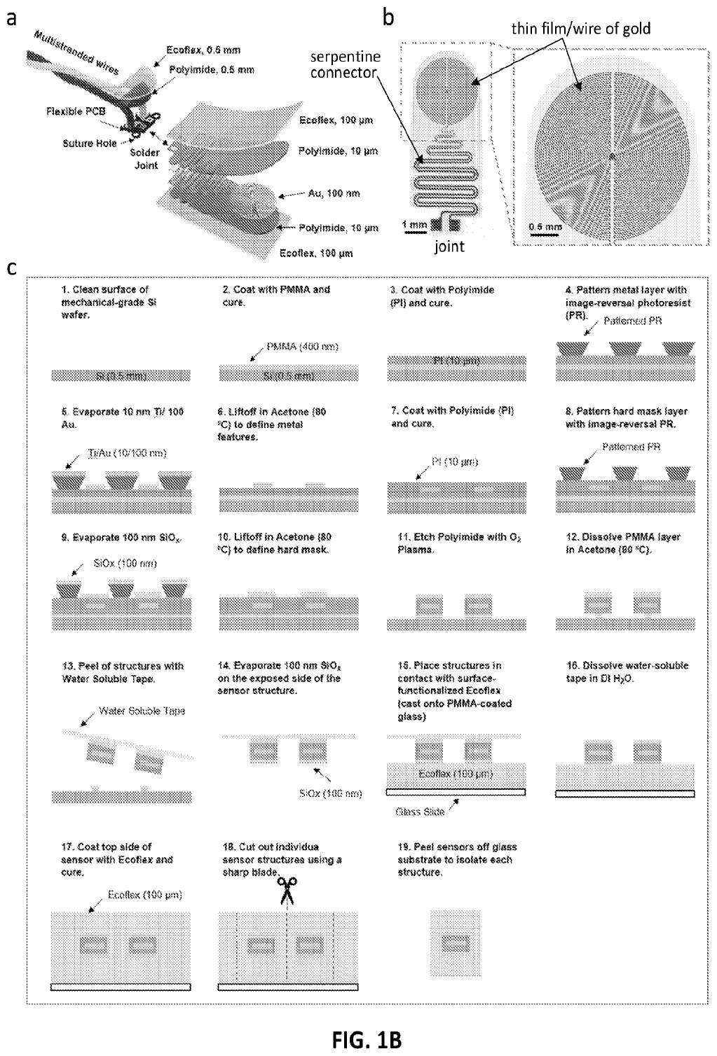

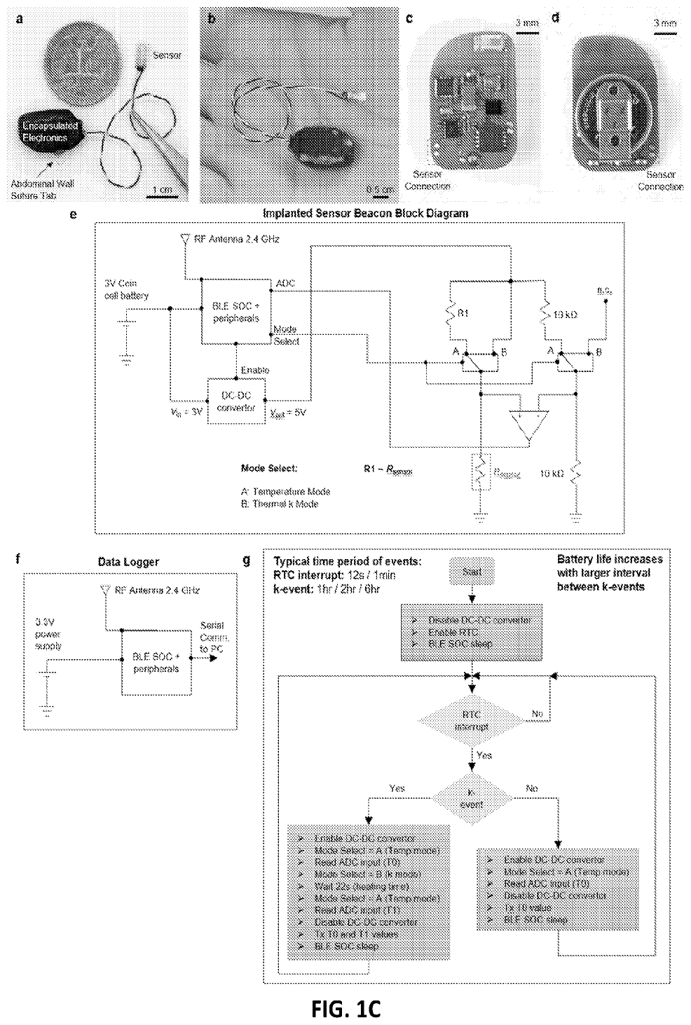

Referring to FIG. 1A, the implantable biosensor comprises a probe operably attached to a target region of a subject; and an electronic module coupled with the probe for wireless, real-time, and continuous measurements of physiological information of the subject.

In one embodiment, the probe includes a temperature sensor and a sensor of thermal conductivity to estimate blood flow, and the electronic module includes a radio module for wirelessly transmitting data to an external receiver; a data process module including an analog to digital front end; and a power module including a source of power. The entire biosensor system is encapsulated to prevent penetration of biofluids, and it is designed to offer soft, flexible mechanical properties to avoid tissue damage. Demonstrated embodiments include both NFC and BLE protocols, and options in battery power and wireless power transfer by inductive coupling.

In some embodiments, the probe is flexible.

In some embodiments, the probe comprises a temperature sensor for measuring the temperature of the target region.

In some embodiments, the probe further comprises a flow sensor for measuring the blood flow of the target region.

In some embodiments, the flow sensor comprises an optoelectronic sensor.

In some embodiments, the optoelectronic sensor comprises one or more light-emitting diodes and one or more one photodiodes.

In some embodiments, the optoelectronic sensor is a photopletismograph sensor.

In some embodiments, the probe further comprises a pressure sensor for measuring pressure.

In some embodiments, the probe further comprises a means for drug delivery.

In some embodiments, the probe has a foot print in a range of about (0.1×0.3)−(0.3×1.0) cm², a thickness in a range of about 50-500 μm, and/or Young's modulus (Y) in a range of about 30-300 kPa.

In some embodiments, the probe is constructed using thin film/wire gold encapsulated by polyimide and silicone layers.

In some embodiments, the electronic module is a flexible, miniaturized electronic module adapted for rechargeable powering, circuit control, signal processing, and wireless data communication.

In some embodiments, the electronic module comprises a flexible printed circuit board (fPCB), electronic components mounted onto the fPCB, and a power module coupled with the electronic components.

In some embodiments, the probe is connected to the fPCB using thin insulated wires and ultrathin stretchable metal serpentine interconnects.

In some embodiments, the fPCB comprises a flexible substrate and conductive traces, pads and outline defined on the flexible substrate.

In some embodiments, the flexible substrate is formed of a flexible material.

In some embodiments, the power module comprises at least one battery.

In some embodiments, the at least one battery is rechargeable.

In some embodiments, the at least one battery is rechargeable via wireless power transfer.

In some embodiments, the electronic components comprise a data processing module coupled to the probe for receiving data from the probe and processing the received data, and a radio module coupled to the data processing module for wireless data transmission to an external device.

In some embodiments, the data processing module comprises a controller and an analog to digital front end.

In some embodiments, the radio module is configured to perform wireless communications using at least one communication protocol of near field communication (NFC), Wi-Fi/Internet, Bluetooth, Bluetooth low energy (BLE), and Cellular communication protocols.

In some embodiments, the radio module comprises at least one of an NFC interface and a Bluetooth interface.

In some embodiments, the external device is a mobile device, a computer, or an ICU monitoring display.

In some embodiments, the device further comprise a customized app with a graphical user interface deployed on the external device that enables real-time visualization, storage, and analysis of measured data. The graphical user interface provides a control interface to the device.

In some embodiments, the device further comprises an elastomeric encapsulation layer at least partially surrounding the probe and the electronic module.

In some embodiments, the elastomeric encapsulation layer is formed of a medical-grade, biocompatible silicone.

In some embodiments, the device includes a radio unit for wirelessly transmitting data to an external receiver, a probe (connected module) that includes a temperature sensor and a sensor of thermal conductivity (to estimate the blood flow), an analog to digital front end, and a source of power.

In some embodiments, the entire device/system is encapsulated to prevent penetration of biofluids.

In some embodiments, it is designed to offer soft, flexible mechanical properties to avoid tissue damage. Demonstrated embodiments include both NFC and BLE protocols, and options in battery power and wireless power transfer by inductive coupling.

In some embodiments, the physiological information comprises tissue temperature, thermal conductivity, and/or blood flow.

In some embodiments, the device is used for continuous, real-time monitoring of organ temperature and perfusion for detecting graft-rejection associated inflammatory processes in organ transplant intra-operatively and/or post-operatively.

In some embodiments, the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

In some embodiments, the thermal power is chosen such that ΔT<2° C.

In some embodiments, the device is mechanically compliant and water resistant.

In some embodiments, when the device is utilized in an animal model of transplantation, the variation of the thermal conductivity can serve as a surrogate marker of kidney allograft rejection. Further, the cortical blood flow could provide important information regarding kidney injury and kidney recovery.

In some embodiments, the device can be utilized in the clinical care of patients for providing continuous information on the thermal conductivity and the blood flow, which allows rapid identification of patients who are at risk of or are in process of having a rejection.

FIGS. 1B-1C respectively shows the details of the probe and its fabrication procedure and the electronic module according to one embodiment of the invention. In the exemplary embodiment, the miniature (0.3×0.7 cm²), soft (Young's modulus (Y) ˜60 kPa), ultrathin (about 220 μm) design of the probe is constructed/fabricated using thin film/wire gold (100 nm) encapsulated by polyimide (10 μm) and silicone (100 μm) layers (panels a-b of FIG. 1). Specifically, as shown in panel c of FIG. 1B, the probe in one embodiment is fabricated in the following steps:
1. cleaning the surface of a mechanical grade silicon (Si) wafer (about 0.5 mm thick);
2. coating a layer of poly(methyl methacrylate) (PMMA) (about 400 nm thick) on the cleaned Si wafer, and curing the coated PMMA layer;
   3. coating a layer of polyimide (PI) (about 10 μm thick) on the PMMA layer and curing the coated PI layer
4. patterning a metal layer on the PI layer with image-reversal photoresist (PR);
5. evaporating 10 nm Ti/100 nm Au on the patterned layer;
6. lifting off in acetone (about 80° C.) to define the metal features;
7. coating the metal features with PI and curing the same;
8. patterning a hard mask layer on the coated metal features with image-reversal PR;
9. evaporating 10 nm SiOₓ on the patterned hard mask layer;
10. lifting off in acetone (about 80° C.) to define the hard mask;
11. etching PI with O₂ plasma;
12. dissolving the PMMA layer in acetone (about 80° C.);
13. peeling of structures with a water soluble tape;
14. evaporating 100 nm SiOₓ on the exposed side of the sensor structure;
15. placing the structure in contact with surface-functionalized Ecoflex (casting onto PMMA-coated glass);
16. dissolving the water soluble tape in DI water (H₂O);
17. coating a top side of the sensor with Ecoflex and curing the same;
18. cuttinh out individual sensor structure; and
19. peeling the sensor off glass substrate to isolate each structure.

It should be noted that the above fabrication procedure, materials and structural sizes of the sensor (probe) is disclosed in accordance with one exemplary embodiment, without intent to limit the scope of the invention, and other fabrication procedures, materials and structural sizes can also be utilized to practice the invention.

In another aspect, the invention relates to a method for continuously monitoring a target region of a subject in real time. The method comprises attaching a device on the target region, wherein the device comprises a probe and an electronic module coupled with the probe for wireless, real-time, and continuous measurements of physiological information of the target region; measuring temperature and perfusion of the target region; and processing the measured temperature and perfusion by the electronic module to identifying a surrogate marker for detecting graft-rejection associated inflammatory processes in organ transplant intra-operatively and/or post-operatively.

In some embodiments, the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

In some embodiments, the thermal power is chosen such that ΔT<2° C.

In some embodiments, said processing the measured temperature and perfusion comprises identifying unique temperature signatures for different rejection-related biological processes/mechanisms.

In some embodiments, the temperature not only provides early warning of rejection episodes but also helps personalized dosing strategies including correct dosing, dosing regimens, and efficacy of different drugs and therapies through monitoring of the magnitude of and time between different features in the temperature including the inflection point, temperature peak, and half-day frequency.

In some embodiments, the surrogate marker is temperature variations on the surface of the target region.

In some embodiments, the method further comprises inferring a degree of damage that occurs during ischemia-reperfusion injury (IRI) and the possible recovery based on the perfusion.

In some embodiments, the method further comprises wirelessly transmitting the processed temperature and perfusion to an external device by the electronic module.

In some embodiments, the method further comprises alerting the subject and/or a physician of possible injury to the graft, based on the surrogate marker.

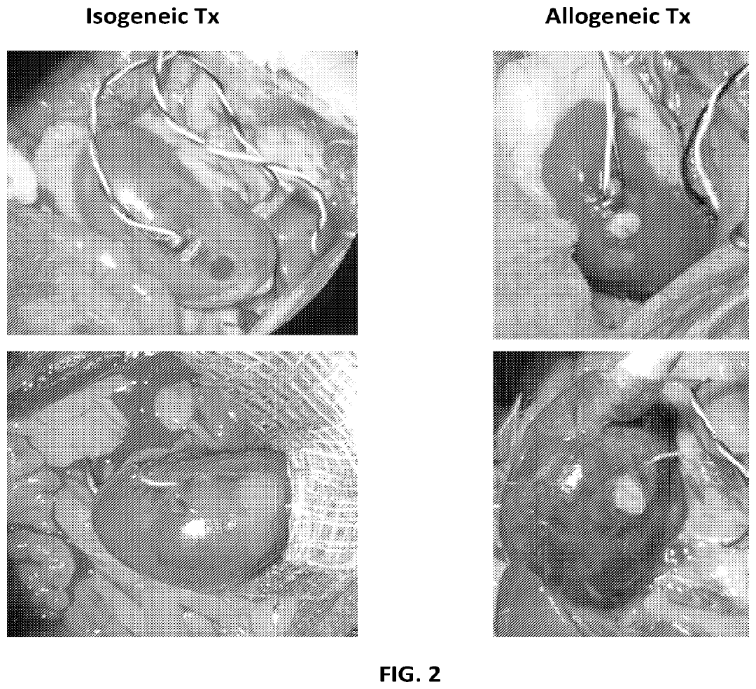

Referring to FIG. 2, the soft, stretchable thermal sensor probe (comprised of ˜100 nm thick gold metal film) is implanted under the kidney capsule. The probe is connected using thin insulated wires and ultrathin stretchable metal serpentine interconnects, to a printed circuit board (PCB) including the sensing and communication circuitry along with the battery to power the device. The pictures visually represent the condition of the transplanted kidney at the start and end of an experiment, for the cases of isogeneic (same donor/recipient inbred rat strains) and allogeneic (different donor/recipient inbred rat strains). For the case of allogeneic transplant, the kidney at the end of the experiment is darker in color and also larger in size.

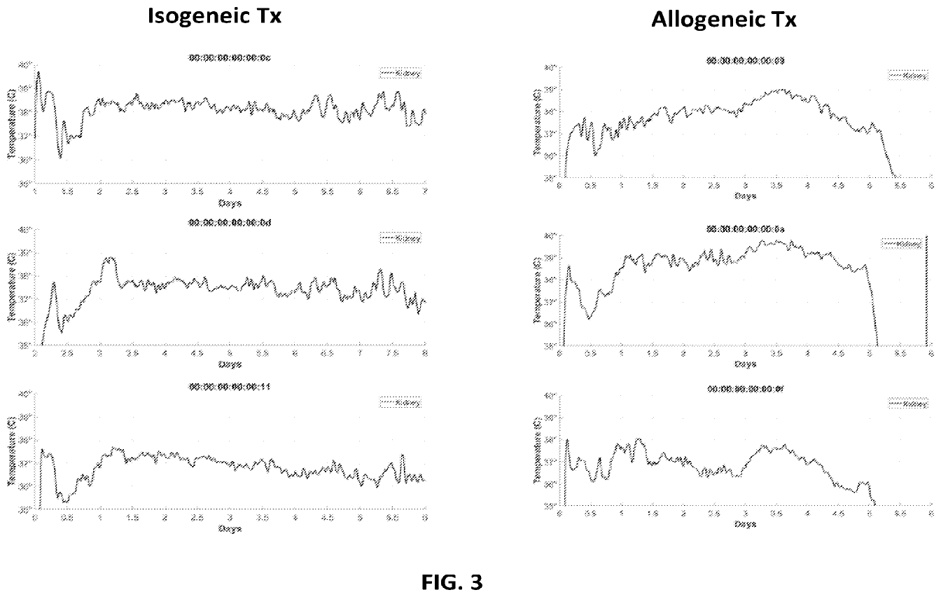

FIG. 3 shows the surface temperatures of the kidney cortex measured by the probe in rat models. Isogeneic transplant temperature data versus time is stable after day 1, with an average value of 37.5° C. For allogeneic transplants, graft rejection is initially observed as a temperature rise seen about day 3 post surgery, with the animal not surviving beyond day 6 post surgery. Severe deterioration in the health of the animal is observed in the sharp decrease in the temperature around day 5-6 for the allogeneic cases.

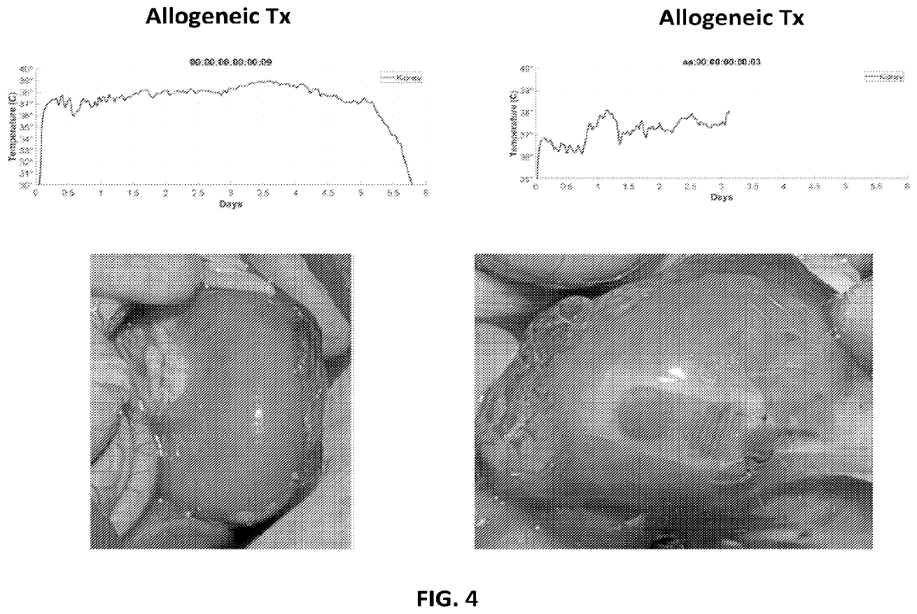

As shown in FIG. 4, to correlate the biological parameters related to transplant rejection with the observed temperature rise from the probe, the animal was sacrificed on day 3 and a nephrectomy of the transplanted kidney was performed to observe tissue histology and blood serum creatinine/blood urea nitrogen collected from blood plasma. Several tissue samples were preserved in formaldehyde and frozen. Extra blood plasma were also saved. Histology of the samples to identify the level of graft rejection is part of ongoing investigation. The pictures show the kidney of the animal sacrificed on day 3 at the time of the sensor explant. Based on tissue histology, another intermediate time point (Day 4 or 5) may be added.

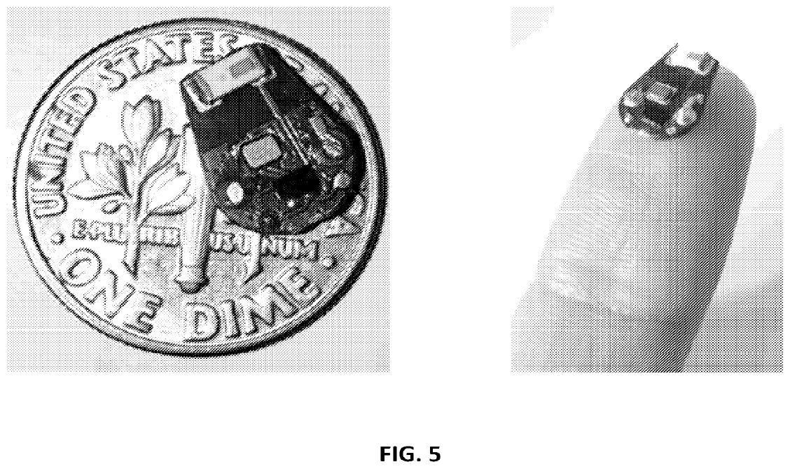

FIG. 5 shows a miniaturized version of the fully implantable thermal sensor is proposed for use in large animal models. The small size of the module allows the entire sensor to fit underneath the kidney capsule of a large mammal, including canine, porcine, and non-human primate models. In some embodiments, the device is about 3 mm thick and about 186 mm³ in volume, and the battery life is a function of sampling rate and battery capacity, and can be typically designed to be about 7-9 months for once an hour sampling rate. The sensor has a resolution of 10 mK.

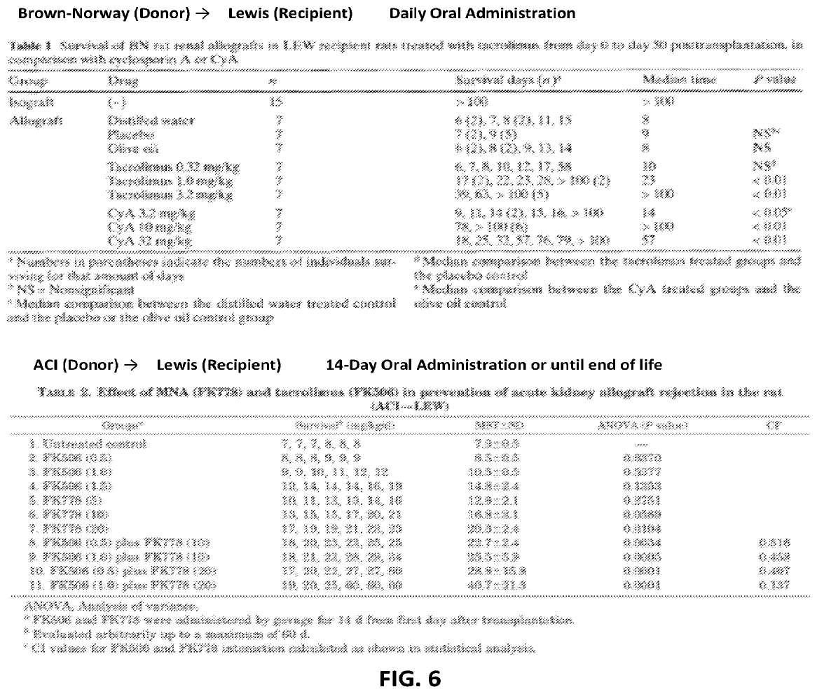

FIG. 6 shows immunosuppressive therapy using FK506 (Tacrolimus) or other commonly used immunosuppressants can prevent/delay the onset of graft rejection beyond the 3-day time point, as observed relative to control groups which do not receive the treatment. The references administered the immunosuppressive therapy using oral gavage. Based on the available information in literature, the plan is to perform allogeneic transplants with immunosuppressive therapy providing a dose of 1 mg/kg daily for a period of 7 days.

Choice of a large animal model for validating device performance NHPs. In the exemplary embodiments, the large animal model of pigs was chosen to validate device functionality and troubleshoot surgical incompatibility, since pain is very easily detectable in pigs, and pigs are readily available. In addition, the current ISO guidelines indicate that the pig is a suitable, if not preferred, model for testing local effects after implantation” for medical devices including stents and mechanical heart valves.

For the future implementations, the following would be considered: complete (2×) more allogeneic Tx for 3-day endpoint, (3×) FK506 experiments with 10-day administration, complete (3×) isogeneic Tx for 3-day endpoint, and collect tissue histology on all samples.

The invention in one aspect provides an implantable biosensor capable of monitoring wirelessly and continuously thermal conductivity and blood flow on the surface of the kidney. According to the invention, real-time thermal conductivity and blood flow changes during kidney allograft rejection can be continuous monitored and detected. Continuous monitoring of temperature variations on the surface of kidney transplants can serve as a surrogate marker for ongoing rejection and immediately alert the patient and the physician of possible injury to the graft. In addition, cortical blood flow can infer the degree of damage that occurs during IRI and the possible recovery.

Among other advantages, the invention improves the understanding of physiologic changes including thermal conductivity and blood flow of the transplanted kidney during kidney allograft rejection, and potentially leads to the implication in humans as a continuous implanted kidney allograft monitor. It is anticipated that this discovery will translate into the clinical care of patients and providing continuous information on thermal conductivity and blood flow will allow rapid identification of patients who are at risk of or are in process of having a rejection.

Internal wireless thermosensor devices and blood flow devices can change the way we practice transplant medicine. This device and future similar devices can provide critical information regarding the health of internal organs (in this case, kidney transplants)

We foresee multiple applications of the invented sensor not only for clinical applications to the field of transplantation but also for monitoring native kidney injuries and kidney recovery, especially during IRI. Currently, there are no approved therapies to prevent IRI in kidneys. This device can provide critical information regarding the health of the kidney and help the physician to make important clinical assessments, clinical decisions, and possible therapeutic interventions.

The economic value of this invention is in our opinion, tremendous as is the impact on the care of our patients.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Continuous Kidney and Kidney Allograft Monitoring Using a Novel Wireless Implanted Sensor

Percutaneous biopsies to detect organ graft rejection are invasive and infrequent. Blood markers are often nonspecific, leading to false negatives and positives. This work features fully implantable thermal sensors capable of continuous, real-time monitoring of kidney temperature and perfusion, for detecting graft-rejection associated inflammatory processes in rat kidney transplant models. The thermal sensors provide early warning of acute kidney transplant rejection relative to blood markers both without (P=0.0016) and with (P=0.0004) immunosuppressant therapy. Changes in blood serum creatinine/urea nitrogen due to rejection lag behind that of kidney temperature (˜2-3 days/2 weeks for cases without/with immunosuppressive therapy).

### INTRODUCTION

Kidney transplant is the preferred treatment for end stage renal disease (ESRD) over dialysis due to increased patient survivability, better quality of life, and cost effectiveness. Over the last decade, about 30% of ESRD patients live with a functioning kidney transplant. Donor kidneys are in limited supply. 78,690 patients were on the waitlist for a kidney transplant at the end of 2019, with median wait times of about 4.3 years. Those able to receive the organ graft often cope with the insidious problem of transplant rejection due to human leukocyte antigen (HLA) genotype mismatch between the donor/recipient. Graft failure can occur at any time: 1-year graft survival is 92.7-97.5%, 5-year graft survival is 77.6-86.6%, and 10-year graft survival drops to 49.5-65.5%.

Early intervention upon recognition of initial stages of rejection could help preserve graft function. The ‘gold standard’ clinical method of detecting transplant rejection occurs through percutaneous renal biopsies, where an about 8 cm needle is inserted through the back for collection of a small sample of kidney cortex tissue for histologic evaluation (using the Banff criteria). This procedure is highly invasive and can come with complications such as bleeding and pain, and less commonly infection and accidental damage to adjacent organs. Thus, biopsies cannot be performed frequently, and are typically only collected on protocol (about 3-12 and 24 months after transplantation) or if blood markers of renal function are elevated. Clinical biomarkers in blood are also used to assess graft health. Specifically, serum creatinine and blood urea nitrogen are diagnostic/predictive indicators of renal transplant viability and can be collected in higher frequency than percutaneous biopsies; however, these markers are nonspecific to rejection and can be modulated by nonrenal factors such as diet, muscle mass, presence of infection, and intake of medications, leading to both false negative and positive indications of rejection. Changes in serum creatinine trail significantly (days-weeks) behind changes in glomerular filtration rate (GRF). There thus remains a strong clinical need for techniques to continuously monitor transplant health for early warning of rejection.

Rat kidney transplant models are essential in studying graft rejection. Previous research in rat kidney transplantation is limited to endpoint histological evaluation or monitoring of kidney function via blood serum at discrete, infrequent times because of maximum limits on blood draw volumes and frequencies. Further, commonly used metrics such as animal survival time and behavioral studies are nonspecific and significant only once rejection has reached an advanced stage. Continuous monitoring of graft health from the moment of transplantation is vital towards detecting rejection especially at the onset or during early stages.

The mechanisms of acute rejection can guide the selection of an appropriate monitoring strategy. Acute rejection can be T-cell mediated or antibody-mediated. T-cell mediated acute rejection is characterized by inflammation of the renal tubules, often the arteries, and accumulation of mononuclear cells in the interstitial spaces. Antibody-mediated acute rejection is characterized by antibodies directed at ABO blood group antigens, putative endothelial antigens, and HLA molecules. Both types of rejection are inflammatory processes. Based on this understanding, thermal measurements of kidney temperature and perfusion could likely detect inflammation associated with rejection.

In this exemplary study, we report fully implantable thermal sensors capable of continuous measurements of kidney temperature and perfusion in rat renal transplant models. To our knowledge, this work is the first demonstration of measurements of temperature and the thermal transport properties of internal organ tissue in vivo, in live, unanesthetized animals with real-time data capture and unrestricted motion. The sensor developed here interfaces directly with the kidney and can detect acute kidney rejection compared to non-rejected grafts. Of greatest significance, our work demonstrates that kidney temperature can be used for early warning of kidney transplant rejection compared to serum creatinine, even when immunosuppressants are administered. This work has tremendous potential for expansion in the field of organ transplantation.

### Results and Discussion

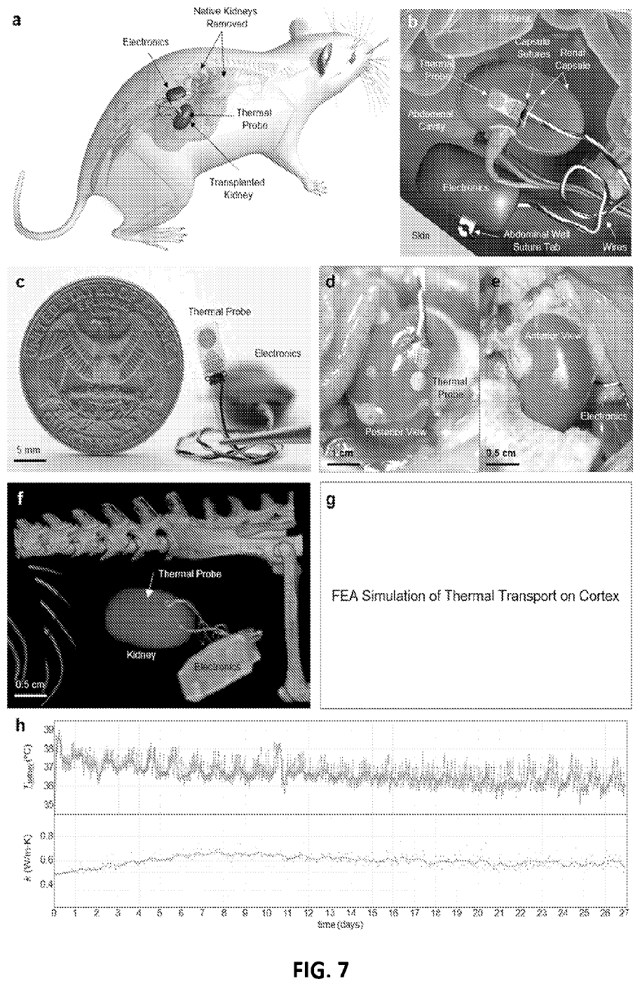

Monitoring Rat Kidney Transplant Rejection using Fully Implantable Thermal Sensors: A kidney transplant model in rats is ideal for thermal sensing research of transplant rejection mechanisms, as it is well-characterized, highly repeatable, low-cost (relative to porcine/non-human primates) and can be studied on short timescales (about 1-4 weeks). The transplanted kidney is grafted distally along the aorta/inferior vena cava on the right side of the body (panels a-b of FIG. 7). Both native kidneys are removed. The thermal sensor lies completely within the abdominal cavity and includes a soft kidney temperature ‘probe,’ connected by wires to an ‘electronics module’ secured to the adjacent abdominal wall. The probe fabrication procedure and electronics details are shown in FIGS. 1B-1C.

The rat kidney is small (about 1×1×2 cm³), soft (Young's modulus (Y)˜4.5 kPa), and highly perfused (288.4±51.3 ml min⁻¹ 100 g tissue⁻¹). The miniature (0.3×0.7 cm²), soft (Y˜60 kPa), ultrathin (about 220 μm) design of the probe constructed using thin film gold (100 nm) encapsulated by polyimide (10 μm) and silicone (100 μm) layers (panel a-b of FIG. 1B and panel c of FIG. 7) interfaces gently and seamlessly with the delicate surface of the kidney without risk of organ damage, such as hemorrhage. The probe directly contacts the dorsal kidney cortex under a tight ‘pocket’ formed under the about 25 μm—thick renal capsule (panels d-e of FIG. 7). The probe is affixed to the renal capsule (panel b of FIG. 7). The thermal sensor implantation occurs in the same procedure as the kidney transplant, without a need for separate surgeries.

The implantation procedure (suture points/type, layout, sensor wire slack) as well as the robust engineering of the thermal sensor allows for real-time continuous data collection in an untethered, freely moving animal, with the sensor remaining in its original position throughout the experimental duration (>2 months for a representative case in panel f of FIG. 7). Data collected using the thermal sensor are kidney temperature (T_{kidney} measured every minute) and cortical perfusion (ω_{kidney} measured every 1-6 hours). T_{kidney} is sensed by measuring changes in resistance of the gold probe (−3 kΩ). Perfusion is measured via thermal anemometry, where ˜1.67 mA current is injected through the 3 mm diameter probe (thermal power q 1.18 mW/mm²) for 22s, causing transient local Joule heating of the kidney tissue by a value ΔT. The magnitude of ΔT depends on ω_{kidney} (panel g of FIG. 7). For the physiologically allowable range of ω_{kidney}/thermal properties of the kidney, the q is chosen such that ΔT<2° C. Finite Element Analysis (FEA) relates ΔT to ω_{kidney}.

Important features, including the impact of the surgery as well as natural biological variations on T_{kidney} and ω_{kidney} are visible in data collected for t=27 days in a control animal's native kidney (with left native kidney nephrectomies) (panel h of FIG. 7). T_{kidney} increases rapidly after the animal is removed from anesthesia (t 0 days) to a peak value ˜3-4 hours post-surgery (˜39° C.). T_{kidney} then falls and settles (concurrently with the release of post-operative analgesia) to an average value of −37.5° C. An irregular pattern in T_{kidney} between t=0-2 days can be attributed to overall surgical recovery and influence from an external heating pad under one side of the animal cage for to assist body temperature regulation (effects of heating pad and room temperature. Room temperature variations do not impact T_{kidney}). After t=2 days, a highly periodic ˜1-day circadian rhythm emerges, which is a direct indicator of the animal's overall good health, ability to recover from the surgery, and efficacy of the sensor design. The implanted sensor does not alter behavior (grooming, activity, food and water consumption, etc.), indicating it is nearly unnoticeable to the animal. Short-term (˜40 mins-1 hr) T_{kidney} variations correlate with motion/activity.

The value of ω_{kidney} increases slowly over t=0-6 days from an initial value to a final value YY. Notably, the value of ω_{kidney} for an animal with both its native kidneys intact is half (YY/2), which are consistent with values in the literature and the understanding that the vascular load of the body is split equally in half between the two kidneys. These observations in control animals provide a point of reference for the key studies presented in this work on transplant and rejection.

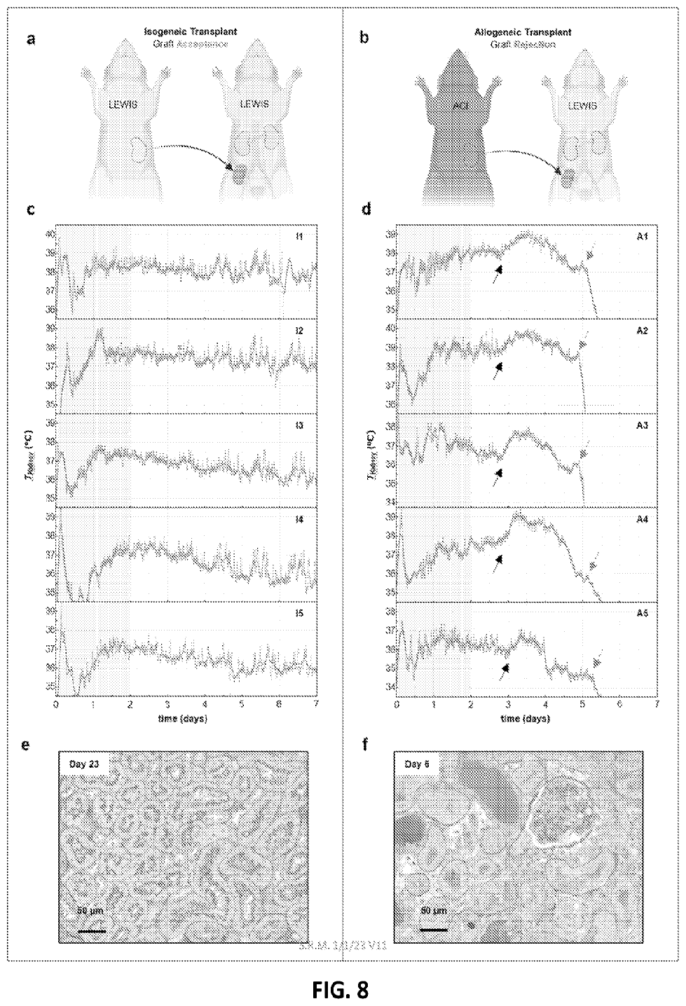

Characterization of Acute Kidney Transplant Rejection: Renal transplantation between inbred rat strains establishes control over transplant acceptance or rejection. Differences in major histocompatibility complex (MHC) and the need for high immune responders guide the choice of rat strains. Isogeneic transplant (panel a of FIG. 8) refers to organ grafts between same-strain inbred rats (in this example, Lewis Rats (MHC haplotype RT1) are both the donors and recipients. Isogeneic transplant is analogous to transplantation between identical twins and results in graft acceptance (indefinite survival time for Lewis-to-Lewis transplants). Allogeneic transplant (panel b of FIG. 8) refers to organ grafts between different strains of inbred rats, where in this example, August-Copenhagen-Irish (ACI) rats (MHC haplotype RT1) are the donors and Lewis rats are the recipients. Allotransplant represents the more clinically relevant case of transplantation in humans, and results in graft rejection without immunosuppressive medication (survival time for ACI-to-Lewis is ˜6 days).

Isograft T_{kidney} undergoes a surgical recovery period (similar to controls shown in panel h of FIG. 7 until t˜3 days due to induced inflammation, effects of analgesia, and post-operative care (panel c of FIG. 8). The circadian rhythm emerges after t˜3 days. The average daily T_{kidney} remains constant after t˜7 days. T_{kidney} trends are similar for all n=5 animals. At the experimental endpoint, minimal adhesions/foreign body response (FBR) appear on the surface of the kidney or around the thermal probe and electronics, indicating the graft is healthy and the probe had no adverse effects.

The measured T_{kidney} for allografts bears little resemblance to that for isografts (panel e of FIG. 8). Post-surgery, at t˜3 days in all allografts (n=5), T_{kidney} rises sharply for ˜12 hours followed by a ˜12-hour fall (the start of this characteristic ‘bump’ is marked by black arrows in panel e of FIG. 8). Around t˜5-6 days, T_{kidney} falls catastrophically to extremely low values (30-32° C.), consistent with behavioral observations of negligible food/water intake and an absence of motion, establishing the experimental endpoint. At this endpoint, enormous adhesions and FBR surrounded an enlarged (˜1.5-2×) graft, which has marbled appearance and necrotic patches distinctive of acute rejection.

It is of significance to note the repeatability of the inflection point at t˜3 days and the T_{kidney} bump observed in all allografts, as well as the similarity of isograft T_{kidney} to controls (with native kidney), which are consistent with the robustness of the rat kidney transplant model. Indefinite normal behavior of isograft recipients shows not only that the kidney is accepted, but also that the sensor size and implant strategy are designed correctly such that the animal does not experience discomfort. This is vital for the welfare of the animals and also to prevent experimental artifacts. Poorly sterilized implants or excessively rough, bulky sensors can inhibit motion, cause severe FBR, and induce stress on the animal.

ω_{kidney} complements the T_{kidney} data and serves as an important indicator of the success of the experiment (i.e., indicator of transplant surgery success). ω_{kidney} is similar to the vascular load for a single kidney, as observed in panel h of FIG. 7 for all 5 isografts and allografts. In addition, ω_{kidney} decreases with time for 3/5 allotransplants, consistent with the severe FBR observed at the endpoint for these particular cases.

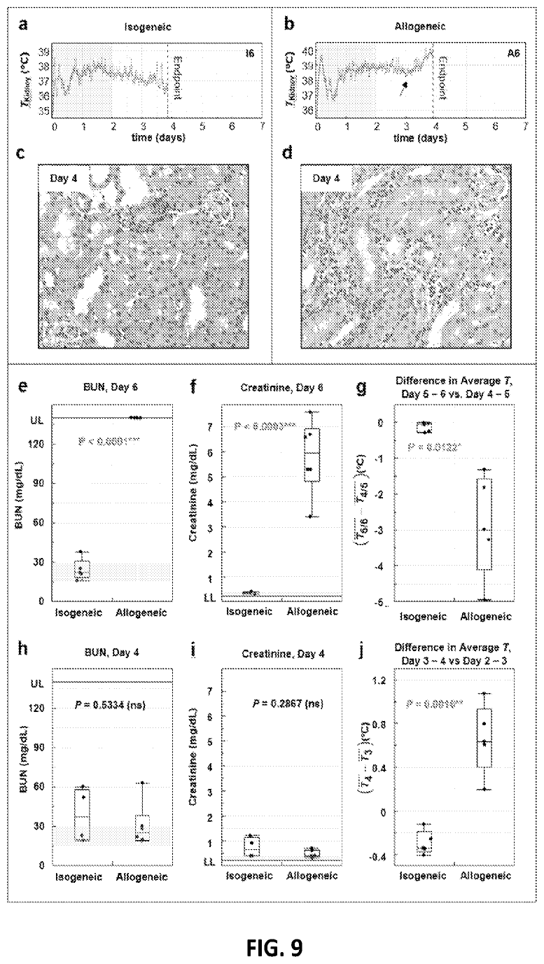

Organ Temperature Provides Early Warning of Transplant Rejection: Comparisons between the key T_{kidney} features observed for allografts and isografts (the inflection point, bump height, and steep temperature decline) and clinical standards for graft function (histology/blood markers) aid in the underlying biological understanding. For simplicity, we refer to the endpoint as t=6 days and the midpoint as t=4 days. At (t˜6 days) of isograft kidney appears normal both macroscopically and in microscopic histological evaluation (panel a of FIG. 9), consistent with the well-known biological understanding that isogeneic grafts are accepted by the body due to genetic similarity of the donor/recipient. In allografts, (t˜5-6 days) histology confirms acute rejection as the cause of graft failure (panel b of FIG. 9). Four out of the five allografts display diffuse cortical necrosis and thrombotic microangiopathy (TMA), characteristic of severe acute rejection. The remaining allograft exhibited Type 1 rejection at t=6 days.

Analysis of blood serum creatinine collected at all endpoints compliment these biopsy results (these data were collected at the endpoint for the experiments in panels c-d of FIG. 8). At t=6 days, isograft BUN and creatinine lie within the normal range for Lewis rats, consistent with the histological evidence of graft acceptance (panels c-d of FIG. 9). In allografts at t=6 days, BUN and creatinine are highly elevated by a factor of >40× (P<0.0001) and ˜7× (P<0.0003), respectively compared to isografts. T̅ₙ̅ denotes the average T_{kidney} between day n and n−1. The temperature decline in allografts, denoted by the difference in average T_{kidney} on the final (T̅₆̅) and penultimate (T̅₅̅) days of survival, is also significant (P=0.0122) (panel e of FIG. 9). Blood markers lend inside to this temperature fall—the enormously high value of BUN suggests that the allografts become uremic toward the endpoint, after the graft has deteriorated significantly. Behavioral characteristics of the allografts at t˜6 days display clear signs of pain and poor health, such as porphyrin staining, slowed or absent motion, and very little no food/water intake. Deterioration in behavior, coupled with blood and histology data illustrate that the t˜6-day timepoint occurs at a very late stage of graft rejection.

Histological assessment of isograft and allograft kidneys harvested between t=3 and 4 days provides important physiological insight into the bump seen in panel d of FIG. 8. We performed a separate set of experiments to harvest allograft and isograft kidneys close to the T_{kidney} inflection point (T_{kidney} and ω_{kidney} data). At t˜4 days, isograft kidneys have normal morphology, while all n=3 allografts exhibited Type I rejection (representative cases in panels f-g of FIG. 9). The case presented in panel d of FIG. 9, harvested at the inflection point (t˜3 days) is that of mild Type 1 rejection, demonstrating that the allograft T_{kidney} bump is coincident with the histological onset of rejection.

Blood serum markers at the midpoint (t=4 days), at the peak of the bump, however, do not offer clear diagnostic value as they do at the endpoint (t=6 days). BUN and serum creatinine on t=4 days for isografts and allografts (n=5) exhibit no significant differences (panels h-i of FIG. 9). At t=4 days, values of BUN and Creatinine in some isografts exceed the normal values for Lewis rats even when histology shows the kidney is normal, likely due to temporary acute kidney injury (AKI) induced by the surgery. Blood markers can, therefore, be nonspecific. On the other hand, the T_{kidney} bump in allografts, provides clinically relevant information. The difference between the T̅₄̅ and T̅₃̅, an indicator of the height of the bump, is significant (˜0.6° C.) for allografts compared to isografts (˜−0.3° C.) (P=0.0016) (panel j of FIG. 9). Animal behavior for allografts at t˜4 days appears normal, indistinguishable from isografts by human observation. This key information reveals that T_{kidney} can be used to detect rejection before loss of graft function is reflected in blood markers or behavior. This data underscores the value of continuous measurements of temperature on the health of the kidney as a means of advanced warning of acute kidney transplant rejection.

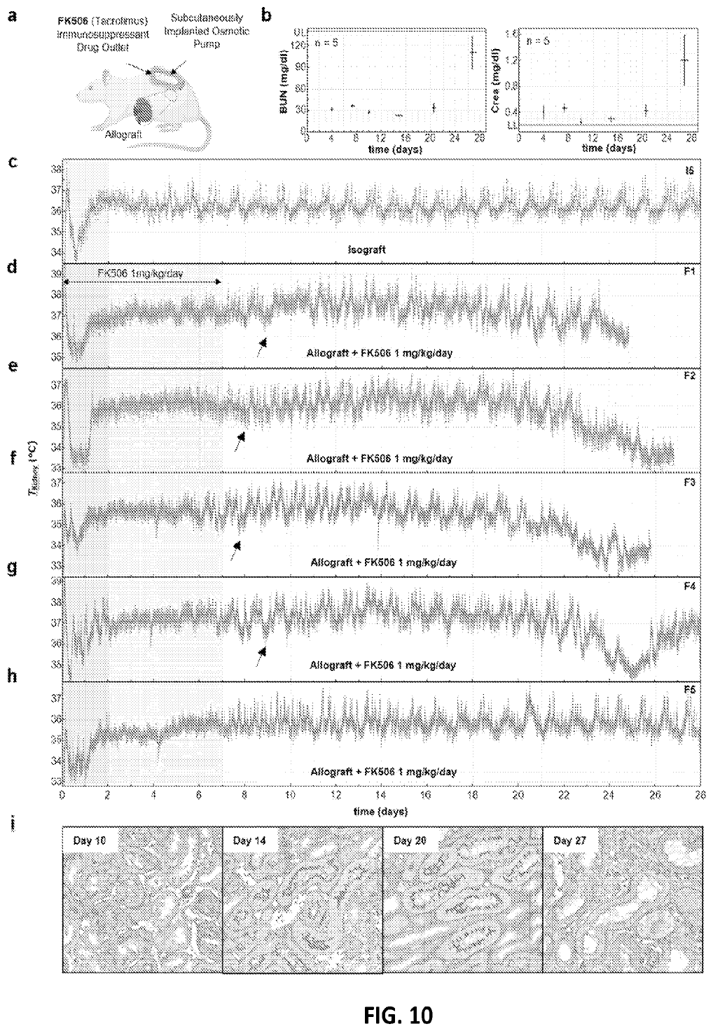

Delayed Graft Rejection with Immunosuppressants: While the previous experiments are important in understanding the relationship between T_{kidney} and underlying transplant acute rejection biology, real-world kidney allotransplants are always treated with immunosuppressants to postpone/inhibit graft rejection. Delaying graft rejection also deconvolves the rejection response from that of surgical recovery. The next set of experiments involved administration of 1 mg/kg/day FK506 (Tacrolimus) for t=0-7 days through a subcutaneously implanted osmotic pump (2ML1, Alzet, Inc.) (panel a of FIG. 10). Continuous delivery has advantages over techniques like oral gavage and injection as drug levels are maintained at a constant level in the body. For these set of experiments, probe data (T_{kidney} and ω_{kidney}) was collected for one case, while the remaining cases made use of a simplified version of the sensor for measurements of T_{kidney}—only using the integrated electronics module. Collection of blood at pre-determined time intervals (t˜4, 7, 10, 14, 21, and 27 days) permits study of the time-evolution of kidney function (panel b of FIG. 10). Creatinine and BUN are elevated above normal levels only for t≥27 days.

Examination of long-term T_{kidney} data collected from an isograft serves as a point of reference for medicated allograft data. Isograft T_{kidney} stabilizes between t=0-3 days then enters a constant average daily T_{kidney} regime with a strong circadian rhythm and natural short-term variations related to motion (panel c of FIG. 10). Allografts treated with 1 mg/kg FK506 have several unique features that are unlike the isograft data (panels d-h of FIG. 10). After the initial t=0-2-day surgical recovery phase, T_{kidney} remains constant for t˜2-7 days, without considerable variations or a circadian cycle, concurrent with the administration period of FK506. For most animals (panels d-g of FIG. 10), a point of inflection at t˜10 days leads to a peak in T_{kidney} at t=14 days. The exact timing of this inflection point varies between animals. After the peak at t=14 days, the temperature slowly decays, and falls catastrophically at t˜22 days for most animals. Medicated allograft T_{kidney} also visually indicates the presence of additional higher frequency components relative to isografts. Long-term data were collected beyond t=28 days.

Histopathology collected before the inflection point, at the T_{kidney} peak, and during the decline lends insight into the corresponding biological activity inside the kidney at these timepoints (corresponding T_{kidney}). The kidney at t˜10 days is not rejected (panel i of FIG. 10). At the peak in temperature (t˜14 days), early-stage Type I acute rejection is observable (panel j of FIG. 10). The morphology of the kidney at t=21 and 27 days (panels k-l of FIG. 10) show progressively worsening signs of Type I or IB acute rejection compared to t=14 days. All five medicated allografts (FK 1 mg/kg) studied for the full lifetime of the graft displayed signs of rejection at the endpoints (panels d-h of FIG. 10).

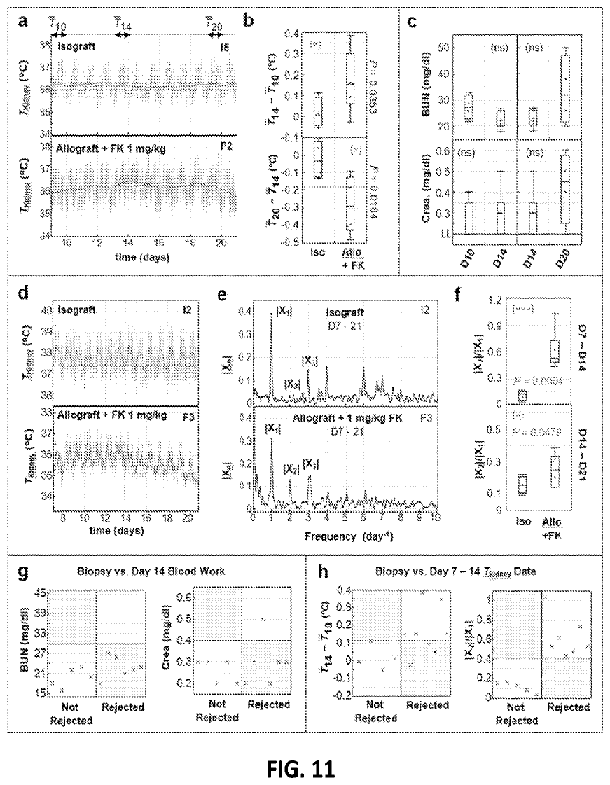

Temperature as a More Reliable Indicator of Acute Rejection Than Blood Markers: We benchmark the early rejection warning capability of features in T_{kidney} for medicated allografts against markers in blood. The first point of interest is the T_{kidney} peak around t˜14 days. T̅₁̅₄̅−T̅₁̅₀̅, a measure of bump height, is appreciable in medicated allografts (median˜0.15° C., P=0.0353) (panels a-b of FIG. 11), but is smaller for the unmedicated case (˜0.6° C.). T̅₂̅₀̅−T̅₁̅₄̅, corresponding to the temperature decrease, is also significant (median ˜−0.3° C., P=0.0184), and is substantially (˜10×) smaller in magnitude compared to the unmedicated case. The medicated allografts also undergo an appreciable change in weight at t˜20 days, indicating deterioration in health. In contrast, blood markers do not exhibit significant differences at these timepoints, even when histological signs of rejection are present (panel c of FIG. 11 and panel i of FIG. 10).

The next feature of interest is the AC component of T_{kidney}, particularly in the range of 10 days<t<14 days, corresponding to the onset of rejection. Medicated allograft time-domain data in this range visibly have frequencies that are not present in isograft data (panel d of FIG. 11). Fast Fourier transform (FFT) analysis of T_{kidney} data reveals the presence of a strong half-day cycle, (ƒ=2 day⁻¹) for all medicated allografts that is not present for isografts. Compared to the magnitude of their respective circadian peaks (|X₁|), the magnitude of the ƒ=2 day⁻¹ rhythm (|X₂|) in the medicated allografts is highly significant between t=10-14 days (P=0.0004) and remains significant between t=14 to 21 days (P=0.0479). The relative magnitude of the ƒ=3 day⁻¹ is also significant for the same time windows. The exact origins of these higher frequency features require additional investigation, we offer a few possible explanations. T-cell activity and/or cellular repair/damage processes are known to be cyclic in nature, synced with the circadian clock and could be a potential root cause of the higher-order frequencies observed in T_{kidney}.

Comparison of measurements of T_{kidney} and Creatinine/BUN to histological evaluation of the kidney is essential in establishing the subclinical relevance of these markers. Confusion matrices illustrate that blood markers (BUN/Creatinine) cannot be used to detect rejection at early timepoints (t=14 days), and almost all medicated allografts are identified as false negatives (panel g of FIG. 11). On the other hand, the T_{kidney} bump correctly identifies rejection at a much higher percentage (75%) of true rejected cases, and the half-day cycle correctly identifies 100% of rejected cases (panel h of FIG. 11). A much larger n is needed to determine true sensitivity and specificity values. These results demonstrate that features in T_{kidney} are more reliable, accurate, and stronger, markers for advanced warning of rejection. BUN/Creatinine appear elevated much later than the onset of rejection.

### CONCLUSION

This interdisciplinary work in engineering and medicine establishes, for the first time, a link between the underlying biology of rejection and its resulting impact on the thermophysical properties of the organ via continuous monitoring of transplant health. The multimodal thermal sensor and analysis presented in this work enables clear identification of rejection (right from the onset) in both unmedicated and medicated allotransplants. Organ temperature is a potential biomarker for transplant health. Our data illustrates that temperature can not only provide early warning of rejection episodes but can also help personalized dosing strategies including correct dosing, dosing regimens, and the efficacy of different drugs and therapies through monitoring of the magnitude of and time between different features in T_{kidney}, (e.g., the inflection point, temperature peak, and half-day frequency). Specialized dose strategies could lower the effective administered dose, potentially reducing common side-effects of immunosuppressants, or increase dosing when signs of rejection appear in the data. Large volumes of experiments are necessary to understand all possible outcomes from rejection and identify unique T_{kidney} signatures for different rejection-related biological processes/mechanisms, including those in chronic rejection. Studies of temperature measurements, and other physical parameters should also be conducted in large animals as their thermoregulatory function is different from small animals, even though the mechanisms of acute rejection are the same. This sensor has potential to detect rejection in for transplantation in other organs, including the liver, heart, lungs, etc. Xenotransplant, another area of growing interest, is another important potential application.

In summary, the invention, among other things, provides novel insights into monitoring of allograft health, calling attention to the importance of continuous measurements, exploration of novel and unconventional biomarkers, and value of implantable sensors.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### REFERENCES

[1]. System, U. S. R. D. 2021 USRDS Annual Data Report: Epidemiology of kidney disease in the United States. (National Institutes of Health, National Institute of Diabetes and Digestive and Kidney Diseases, Bethesda, MD).
[2]. Lo, D. J., Kaplan, B. & Kirk, A. D. Biomarkers for kidney transplant rejection. Nat Rev Nephrol 10, 215-225, doi:10.1038/nrneph.2013.281 (2014).
[3]. Bakdash, K. et al. Complications of Percutaneous Renal Biopsy. Semin Intervent Radiol 36, 97-103, doi:10.1055/s-0039-1688422 (2019).
[4]. Edelstein, C. L. in Biomarkers of Kidney Disease (Second Edition) (ed Charles L. Edelstein) 241-315 (Academic Press, 2017).
[5]. Bilolo, K. K. et al. Synergistic effects of malononitrilamides (FK778, FK779) with tacrolimus (FK506) in prevention of acute heart and kidney allograft rejection and reversal of ongoing heart allograft rejection in the rat. Transplantation 75, 1881-1887, doi:10.1097/01.TP.0000064710.78335.D3 (2003).
[6]. Jiang, H. et al. Tacrolimus versus cyclosporin A: a comparative study on rat renal allograft survival. Transpl Int 12, 92-99, doi:10.1007/s001470050192 (1999).
[7]. Cornell, L. D., Smith, R. N. & Colvin, R. B. Kidney transplantation: mechanisms of rejection and acceptance. Annu Rev Pathol 3, 189-220, doi:10.1146/annurev.pathmechdis.3.121806.151508 (2008).
[8]. Singh, N., Pirsch, J. & Samaniego, M. Antibody-mediated rejection: treatment alternatives and outcomes. Transplant Rev (Orlando) 23, 34-46, doi:10.1016/j.trre.2008.08.004 (2009).
[9]. Gennisson, J.-L. et al. Multiwave technology introducing shear wave elastography of the kidney: Preclinical study on a kidney fibrosis model and clinical feasibility study on 49 human renal transplants. Proceedings—IEEE Ultrasonics Symposium, 1356-1359, doi:10.1109/ULTSYM.2010.5935793 (2010).
[10]. Romero, C. A. et al. Noninvasive measurement of renal blood flow by magnetic resonance imaging in rats. Am J Physiol Renal Physiol 314, F99-F106, doi:10.1152/ajprenal.00332.2017 (2018).
[11]. Raman, R. N. et al. Evaluation of the contribution of the renal capsule and cortex to kidney autofluorescence intensity under ultraviolet excitation. J Biomed Opt 14, 020505, doi:10.1117/1.3094948 (2009).

1. A device implantable for continuously monitoring a target region of a subject in real time, comprising:
a probe operably attached to the target region; and
an electronic module coupled with the probe for wireless, real-time, and continuous measurements of physiological information of the target region.

2. The device of claim 1, wherein the probe is flexible.

3. The device of claim 2, wherein the probe comprises a temperature sensor for measuring the temperature of the target region.

4. The device of claim 3, wherein the probe further comprises a flow sensor for measuring the blood flow of the target region.

5. The device of claim 4, wherein the flow sensor comprises an optoelectronic sensor.

6. The device of claim 5, wherein the optoelectronic sensor comprises one or more light-emitting diodes and one or more one photodiodes.

7. The device of claim 5, wherein the optoelectronic sensor is a photopletismograph sensor.

8. The device of claim 4, wherein the probe further comprises a pressure sensor for measuring pressure.

9. The device of claim 4, wherein the probe further comprises a means for drug delivery.

10. The device of claim 2, wherein the probe has a foot print in a range of about (0.1×0.3)−(0.3×1.0) cm², a thickness in a range of about 50-500 μm, and/or Young's modulus (Y) in a range of about 30-300 kPa.

11. The device of claim 10, wherein the probe is constructed using thin film/wire gold encapsulated by polyimide and silicone layers.

12. The device of claim 1, wherein the electronic module is a flexible, miniaturized electronic module adapted for rechargeable powering, circuit control, signal processing, and wireless data communication.

13. The device of claim 12, wherein the electronic module comprises a flexible printed circuit board (fPCB), electronic components mounted onto the fPCB, and a power module coupled with the electronic components.

14. The device of claim 13, wherein the probe is connected to the fPCB using thin insulated wires and ultrathin stretchable metal serpentine interconnects.

15. The device of claim 13, wherein the fPCB comprises a flexible substrate and conductive traces, pads and outline defined on the flexible substrate.

16. The device of claim 13, wherein the flexible substrate is formed of a flexible material.

17. The device of claim 13, wherein the power module comprises at least one battery.

18. The device of claim 16, wherein the at least one battery is rechargeable.

19. The device of claim 16, wherein the at least one battery is rechargeable via wireless power transfer.

20. The device of claim 13, wherein the electronic components comprise a data processing module coupled to the probe for receiving data from the probe and processing the received data, and a radio module coupled to the data processing module for wireless data transmission to an external device.

21. The device of claim 20, wherein the data processing module comprises a controller and an analog to digital front end.

22. The device of claim 20, wherein the radio module is configured to perform wireless communications using at least one communication protocol of near field communication (NFC), Wi-Fi/Internet, Bluetooth, Bluetooth low energy (BLE), and Cellular communication protocols.

23. The device of claim 20, wherein the radio module comprises at least one of a near-field communication (NFC) interface and a Bluetooth interface.

24. The device of claim 20, wherein the external device is a mobile device, a computer, or an ICU monitoring display.

25. The device of claim 20, further comprising a customized app with a graphical user interface deployed on the external device that enables real-time visualization, storage, and analysis of measured data, wherein the graphical user interface provides a control interface to the device.

26. The device of claim 20, further comprising an elastomeric encapsulation layer at least partially surrounding the probe and the electronic module.

27. The device of claim 26, wherein the elastomeric encapsulation layer is formed of a medical-grade, biocompatible silicone.

28. The device of claim 1, wherein the target region is an organ or transplanted organ.

29. The device of claim 28, wherein the organ or transplanted organ is a kidney, a liver, a lung, a heart or other organ.

30. The device of claim 28, wherein the physiological information comprises tissue temperature, thermal conductivity, and/or blood flow.

31. The device of claim 30, being used for continuous, real-time monitoring of organ temperature and perfusion for detecting graft-rejection associated inflammatory processes in organ transplant intra-operatively and/or post-operatively.

32. The device of claim 31, wherein the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

33. The device of claim 32, wherein the thermal power is chosen such that ΔT<2° C.

34. The device of claim 1, being mechanically compliant and water resistant.

35. A method for continuously monitoring a target region of a subject in real time, comprising:
attaching a device on the target region, wherein the device comprises a probe and an electronic module coupled with the probe for wireless, real-time, and continuous measurements of physiological information of the target region;
measuring temperature and perfusion of the target region; and
processing the measured temperature and perfusion by the electronic module to identifying a surrogate marker for detecting graft-rejection associated inflammatory processes in organ transplant intra-operatively and/or post-operatively.

36. The method of claim 35, wherein the temperature is measured by measuring changes in resistance of the probe, and the perfusion is measured via thermal anemometry, wherein current is injected through the probe with a thermal power, causing transient local Joule heating of the target region tissue by a value of temperature change, ΔT, wherein the magnitude of ΔT depends on the perfusion.

37. The method of claim 36, wherein the thermal power is chosen such that ΔT<2° C.

38. The method of claim 35, wherein said processing the measured temperature and perfusion comprises identifying unique temperature signatures for different rejection-related biological processes/mechanisms.

39. The method of claim 38, wherein the temperature not only provides early warning of rejection episodes but also helps personalized dosing strategies including correct dosing, dosing regimens, and efficacy of different drugs and therapies through monitoring of the magnitude of and time between different features in the temperature including the inflection point, temperature peak, and half-day frequency.

40. The method of claim 39, wherein the surrogate marker is temperature variations on the surface of the target region.

41. The method of claim 35, further comprising inferring a degree of damage that occurs during ischemia-reperfusion injury (IRI) and the possible recovery based on the perfusion.

42. The method of claim 35, further comprising wirelessly transmitting the processed temperature and perfusion to an external device by the electronic module.

43. The method of claim 35, further comprising alerting the subject and/or a physician of possible injury to the graft, based on the surrogate marker.

44. The method of claim 35, wherein the target region is an organ or transplanted organ.

45. The method of claim 44, wherein the organ or transplanted organ is a kidney, a liver, a lung a heart, or other organ.
